(19) United States
(12) Patent Application Publication
(10) Pub. No.: US 2023/0000958 A1
(43) Pub. Date: Jan. 5, 2023

(54) Immune Booster Food Supplement

(71) Applicant: Daaniel Karnwie-Tuah, Santa Clarita, CA (US)

(72) Inventor: Daaniel Karnwie-Tuah, Santa Clarita, CA (US)

(21) Appl. No.: 17/305,074

(22) Filed: Jun. 30, 2021

(51) Int. Cl. A61K 38/48; A61K 31/375; A61K 31/593; A61K 33/30; A61K 33/00; A61K 47/46; A61K 9/00; A23L 33/115; A23L 33/21; A23L 33/17; A23L 33/105; A23L 33/16; A23L 33/155; A23L 29/00; A23L 33/00

(52) U.S. Cl. CPC A61K 38/4873; A61K 31/375; A61K 31/593; A61K 33/30; A61K 33/00; A61K 47/46; A61K 9/0056; A23L 33/115; A23L 33/21; A23L 33/17; A23L 33/105; A23L 33/16; A23L 33/155; A23L 29/06; A23L 33/40; A23V 2002/00

(57) ABSTRACT

The present invention discloses a capsule supplement that can boost the immune system and offer advanced protection. The food supplement is a food composition for oral administration as an anti-inflammatory and immune booster. The invention achieves the results by using its anti-inflammatory properties, fats, carbohydrates, protein, fruits, minerals and vitamins, antioxidant and is fortified with: vitamin C, vitamin D, zinc, and bromelain. It improves overall health and wellness. The said supplement increases the body defenses against viruses and other illnesses. Unlike therapeutic interventions, this supplement composes of ingredients that are in the food category, which is the primary source of nutrients for the human body. It helps the body maintains its healthy normal functions by using its natural source of sustenance.

### TECHNICAL FIELD

The present invention relates to food supplements, and particularly to an immune booster food supplement formulated to improve immune system and offer advanced protection as well as improving the overall human health.

### BACKGROUND ART

The supplements currently available are used in combinations to achieve similar results. For example, multivitamin, and many other individual extracts are used combinations because no single one is effective enough to provide the improvement needed to achieve the results. The currently available supplements that support the immune function rely on heavily on the combination of regular vitamins in addition to few fruits or vegetables that have little to do with the immune system.

A study carried by NIH researchers reveals that “Vitamin C in humans must be ingested for survival. Vitamin C is an electron donor, and this property accounts for all its known functions. As an electron donor, vitamin C is a potent water-soluble antioxidant in humans. Antioxidant effects of vitamin C have been demonstrated in many experiments in vitro. Human diseases such as atherosclerosis and cancer might occur in part from oxidant damage to tissues.” Padayatty S J, et al., “Vitamin C as an antioxidant: evaluation of its role in disease prevention,” J Am Coll Nutr. February 2003; 22(1):18-35.

The immune system's role is to defend the body against external and internal aggression. These attacks penetrate at the level of trade surfaces or lesions in the natural barriers of protection. The biological attacks are caused by foreign organisms, pathogenic or not, such as microorganisms (bacteria, fungi), viruses or parasites (protozoa, helminths). The internal aggressions are the fact of cells become tumorous or whose function puts directly or indirectly the normal components of the organism (the self) in danger. The aging of the immune system is characterized by thymic involution, which begins at puberty and is almost complete at middle age. This involution results in a reduction of the size of the thymus and a progressive replacement of the lymphocyte and vascular space by adipose tissue.

Further, the pandemic has been accelerating demand for immunity boosting products and ingredients. Functional ingredients suppliers have revealed that the coronavirus has caused 75 percent of consumers to seek out healthier foods and beverages, and according to Google Trends data, searches for the combined terms, ‘food’ and ‘immune system’ skyrocketed by 670 percent between February and March, 2021. But, while COVID-19 is piquing consumer interest in a variety of trendy products, not all are worth the hype. The following are the most popular trends in the immunity-boosting market with the scientific evidence, or lack thereof, to support their claims. Wellness shots are small servings of concentrated juices consisting of ingredients that are considered to have beneficial properties. Immune boosting wellness shots typically have one or more of a number of ingredients such as ginger, turmeric, wheatgrass, citrus, echinacea, cayenne pepper or apple cider vinegar. Global consumer surveys conducted by InsightsNow, Inc. before and during the pandemic reveal that COVID-19 has affected consumer perceptions of health and wellness, creating a renewed interest in functional foods or foods that are fortified, enriched or enhanced, providing health benefits beyond the provision of basic nutrients.

A few prior art exists in this field of technology.

A French patent number FR2882896A1 filed in 2005 by LARENA S A discloses a supplementary food composition, useful e.g. to treat and/or prevent sarcopenia, comprises proteins rich in branched amino acids, ginsenosides, zinc, selenium, vitamins (B complex, D, C and E), algal extract, polyphenols and carotenoids. It claims 1. Substitute food composition comprising at least natural proteins rich in branched-chain amino acids, amino acids such as arginine and glutamine, ginsenosides, zinc, selenium, vitamins B1, B2, B3, B5, B6, B8, B9 and B12, vitamin D, vitamin C, vitamin E, seaweed extract, polyphenols and carotenoids. However, although, it claims the use of Vitamin C, Vitamin D, Zinc and an antioxidant, it does not claim the use of bromelain.

U.S. patent number US20080095757A1 filed in 2007 by FRUITFUL YIELD Inc discloses use of Vitamin C in a composition. It claims, 1. A composition comprising ascorbate and at least one ascorbate-glucose transport enhancer. 2. The composition of claim 1, wherein the ascorbate is in the form of vitamin C, ascorbic acid, L-ascorbic acid, L-xylo-ascorbic acid, L-threo-hex-2-enoic acid γ-lactone, an ascorbyl ester, ascorbyl palmitate, an ascorbyl phosphate ester, a reacted or blended mineral ascorbate, dehydroascorbate, or a vitamin C metabolite, and 3. The composition of claim 2, wherein the mineral ascorbate is calcium ascorbate, magnesium ascorbate, zinc ascorbate, sodium ascorbate, or potassium ascorbate. Even if it discloses and claims the use of Vitamin C, Zinc, Bromelain and an antioxidant, it fails to teach about the use of Vitamin D.

It is now clear that the above supplements currently available are used in combinations to achieve similar results. For example, multivitamin, and many other individual extracts are used combinations because no single one is effective enough to provide the improvement needed to achieve the results. Therefore, there is need to develop a more effective food supplement that can boost human immunity against many diseases including COVID-19.

### OBJECTIVES OF THE INVENTION

It is an object of the present invention to provide a supplement that can protect body by its ability to fight harmful bacteria and viruses, protect cells from the damage and improve healing. A single capsule performs many functions that are usually associated with several different supplements or medications.

The other object of the present invention is to avail a supplement that increases the body defenses against viruses and other illnesses.

### SUMMARY OF THE INVENTION

The following summary is an explanation of some of the general inventive steps for the materials, method, and apparatus in the description. This summary is not an extensive overview of the invention and does not intend to limit the scope beyond what is described and claimed as a summary.

In the first aspect, the present invention discloses an immune booster food supplement, comprising fats, carbohydrates, protein, fruits, minerals and vitamins, antioxidant and fortified with: vitamin C, vitamin D, sodium, zinc, and bromelain. The said capsule supplement can boost the immune system and offer advanced protection.

In another aspect, the present invention discloses a food supplement in form of a composition for oral administration as an anti-inflammatory and immune booster by using anti-inflammatory properties of fats, carbohydrates, protein, fruits, minerals and vitamins, antioxidant and is fortified with: vitamin C, vitamin D, zinc, and bromelain.

In a third aspect of the present invention is to improve overall health and wellness by providing a supplement that composes of ingredients that are in the food category, which is the primary source of nutrients for the human body, unlike therapeutic interventions. Unlike therapeutic interventions, this supplement composes of ingredients that are in the food category, which is the primary source of nutrients for the human body. It helps the body maintains its healthy normal functions by using its natural source of sustenance.

### DETAILED DESCRIPTION OF THE INVENTION

Studies have shown that a significant number of Americans do not consume sufficient amounts of Vitamins C and D. Most of their daily diet lack Zinc. They rarely consume adequate servings of fruits and vegetables including pineapple, ginger, lemon, lime and bromelain. Increasing the amount of these components consumed in a standard daily diet is one way to rectify this deficiency. Dietary supplements are another option, and there are a number of commercially available vitamin C and vitamin D supplements in the market. The effectiveness of such supplements is limited, however, by the absorption rates and transport efficiencies of the body.

In one embodiment, the present invention describes an immune booster food supplement, comprising fats, carbohydrates, protein, fruits, minerals and vitamins, antioxidant and fortified with: vitamin C, vitamin D, sodium, zinc, and bromelain. The said supplement is as a capsule that boost the immune system and offer advanced protection. It achieves the results by using its anti-inflammatory properties, fats, carbohydrates, protein, fruits, minerals and vitamins, antioxidant and is fortified with: vitamin C, vitamin D, zinc, and bromelain which improves overall health and wellness.

In another embodiment, the invention reveals a supplement that contains the blend of gluten free, allergen free, non-GMO, natural ingredients comprising their unique functions as follows:

Vitamin C is an antioxidant. It protects your cells from the damage caused by free radicals, improves your the immune system, and supports your body's healing process.

Vitamin D helps the immune system to fight off invading bacteria and viruses, contains the required nutrients for good health, helps your body to absorb calcium, a building blocks for strong bones, protects you from developing osteoporosis, helps your nerves to carry messages between your brain and your body, prevents heart disease and high blood pressure and prevents cancer, such as colon, prostate and breast cancers.

Zinc helps the immune system fight off invading bacteria and viruses, helps your metabolism function, supports your sense of taste and smell and helps with wound healing.

Pineapple is loaded with antioxidants and helps digestion, anti-Inflammatory and speeds recovery after surgery or exercise. It fortifies the immune body functions.

Potassium is equally vital to the body functions.

Ginger is used to fight harmful bacteria and viruses. It is an anti-inflammatory and antioxidant. It helps in weight management, reduces risk of heart disease and reduces LDL (bad) cholesterol

Lemon has high Vitamin C content. It is also used in weight management and reduces the risk of heart diseases, Anaemia, Kidney stones, digestive issues, and cancer.

Bromelain acts as an anti-inflammatory and improves your allergies, indigestion, supports your body healing process and promotes the overall health.

Lime has high antioxidants. It improves your immune system, reduces your heart disease risk factors, prevents kidney stones, aids in iron absorption and promotes healthy skin.

Compositions disclosed herein include compounds with anti-inflammatory properties, minerals and vitamins, antioxidant and is fortified with: vitamin C, vitamin D, zinc, and bromelain. The preferred amounts of are depicted in the table below showing Example 1:

### EXAMPLE 1: FOOD SUPPLEMENT COMPOSITION

60 Serving per Container: Serving size 1 Capsule

| | | Amount per Serving | | % Daily |
|---|---|---|---|---|
| No | | in Calories | | Value Intake |
| 1 | Total Fat | <1 | g | <1% |
| | Saturated Fat | <1 | g | ± |
| | Trans Fat | <1 | g | <1% |
| 2 | Cholesterol | <1 | g | <1% |
| | Sodium | 1.0 | mg | 0% |
| 3 | Total Carbohydrate | <1 | g | <1% |
| | Dietary Fiber | <1 | g | <1% |
| | Total Sugars | <1 | g | <1% |
| | Includes added Sugars | <1 | g | — |
| 4 | Protein | <1 | g | <1% |
| 5 | Vitamin C (as Ascorbic Acid) | 40.0 | mg | 44% |
| 6 | Vitamin D3 (as Cholescalciferol) | 12.5 | mcg | 63% |
| 7 | Zinc (as Zinc Oxide) | 25 | mg | 227% |
| 8 | Bromelain 600 GDU | 100.0 | mg | — |
| 9 | Pineapple 200 Sieve | 100.0 | mg | ± |
| 10 | Ginger Powder | 100.0 | mg | ± |
| 11 | Lime Powder | 100.0 | mg | ± |
| 12 | Lemon Powder | 120.0 | mg | ± |

NB: The % Daily Value (DV) are based on a 2,000 Calorie Diet.
± Daily Value not established

In at least one particularly preferred embodiment, a composition includes Vitamin C in an amount from about 34% of daily intake by weight of actives to about 54% of daily intake by weight of actives and at least Vitamin D3 in an amount from about 53% of daily intake of by weight of actives to about 73% of daily intake by weight of actives. Zinc in an amount from about 217% of daily intake by weight of actives to about 137% of daily intake by weight of actives. Pineapple in an amount from about 90 mg by weight of actives to about 110 mg by weight of actives. Ginger powder in an amount from about 90 mg by weight of actives to about 110 mg by weight of actives. Lime powder in an amount from about 90 mg by weight of actives to about 110 mg by weight of actives. Lemon powder in an amount from about 100 mg by weight of actives to about 120 mg by weight of actives.

Dietary fibres in an amount below <1 g or daily intake of <1% by weight of actives. Saturated fats in an amount below <1 g or daily intake of <1% by weight of actives. Proteins in an amount below <1 g or daily intake of <1% by weight of actives. Saturated fats in an amount below <1 g or daily intake of <1% or daily intake of <1% by weight of actives. The total weight of actives is determined by the total weight of all compositional components providing Vitamin C and D and all compositional components. The total weight percentages of all components providing nutrients and other components of a composition should thus equal 100%. Compositions disclosed herein can be provided in any suitable dosage form. Preferably compositions are provided in an oral dosage form or a topical dosage form. For example, compositions can be in a dosage form that is a powder, a microencapsulated powder, granules, a granulated powder, a capsule, a softgel capsule, a two-piece capsule, a pre-blended mixture of ingredients, or a blended mixture of ingredients in the capsule.

In a final embodiment, the invention provides a use of the above immune booster food supplement composition for the preparation of a food composition, a food supplement, or a dietary product for the prevention and/or treating declining immune system in the elderly, sarcopenia, preventing the decline of the immune system, preventing immunoscenescence, preventing the decline of cellular immune responses, preventing and/or reducing the number of infectious episodes, to restore antioxidant status in the elderly.

### INDUSTRIAL APPLICATION

The present invention applies to food industry. The invention provides a supplement that has the ability to fight harmful bacteria and viruses. It has the capacity to protect cells from the damage and improve healing. A single capsule performs many functions that are usually associated with several different supplements or medications.

The supplement increases the body defenses against viruses and other illnesses. Unlike therapeutic interventions, this supplement composes of ingredients that are in the food category, which is the primary source of nutrients for the human body. It helps the body maintains its healthy normal functions by using its natural source of sustenance.

What is claimed is:

1. An immune booster food supplement, comprising fats, carbohydrates, protein, fruits, minerals and vitamins, antioxidant and fortified with: vitamin C, vitamin D, sodium, zinc, and bromelain

2. The immune booster food supplement according to claim 1, wherein the vitamin C is in an amount from about 34% of daily intake by weight of actives to about 54% of daily intake by weight of actives.

3. The immune booster food supplement according to claim 1, wherein the vitamin D3 is in an amount from about 53% of daily intake of by weight of actives to about 73% of daily intake by weight of actives.

4. The immune booster food supplement according to claim 1, wherein the zinc is in an amount from about 217% of daily intake by weight of actives to about 137% of daily intake by weight of actives.

5. The immune booster food supplement according to claim 1, wherein the pineapple in an amount from about 90 mg by weight of actives to about 110 mg by weight of actives.

6. The immune booster food supplement according to claim 1, wherein the ginger powder is in an amount from about 90 mg by weight of actives to about 110 mg by weight of actives.

7. The immune booster food supplement according to claim 1, wherein the lime powder is in an amount from about 90 mg by weight of actives to about 110 mg by weight of actives.

8. The immune booster food supplement according to claim 1, wherein the lemon powder in an amount from about 100 mg by weight of actives to about 120 mg by weight of actives.

9. The immune booster food supplement according to claim 1, wherein the carbohydrate is in the form of dietary fibres.

10. The immune booster food supplement according to claim 9, wherein the dietary fibres is in an amount below <1 g or daily intake of <1% by weight of actives.

11. The immune booster food supplement according to claim 1, wherein the fat is in the form of saturated fats.

12. The immune booster food supplement according to claim 11, wherein the saturated fats saturated fats is in an amount below <1 g or daily intake of <1% by weight of actives.

13. The immune booster food supplement according to claim 1, wherein the protein is in an amount below <1 g or daily intake of <1% by weight of actives.

14. The immune booster food supplement according to claim 1, wherein the sodium is in an amount below <1 g or daily intake of <1% or daily intake of <1% by weight of actives.

15. An immune booster food supplement, wherein the total weight of actives is determined by the total weight of all compositional components fortified with Vitamins C and D and all compositional components further comprising at least fats, carbohydrates, protein, fruits, minerals and vitamins, antioxidant and, sodium, zinc, and bromelain.

16. The immune booster food supplement according to claim 15, wherein the total weight percentages of all components providing nutrients and other components of a composition should thus equal 100%.

17. The immune booster food supplement according to claim 16, wherein the compositions is presented in any suitable dosage form.

18. The immune booster food supplement according to claim 17, wherein the compositions are preferably provided in an oral dosage form.

19. The immune booster food supplement according to claim 18, wherein the oral dosage form is in form of a capsule.

20. The immune booster food supplement according to claim 19, wherein the capsule is in form is a powder, a microencapsulated powder, granules, a granulated powder, a soft gel capsule, a two-piece capsule, a pre-blended mixture of ingredients, or a blended mixture of ingredients in the capsule.

21. The use of an immune booster food supplement composition as described in any one of claims 1 to 20, for the preparation of a food composition, a food supplement, or a dietary product for the prevention and/or treating declining immune system in the elderly, sarcopenia, preventing the decline of the immune system, preventing immunoscenescence, preventing the decline of cellular immune responses, preventing and/or reducing the number of infectious episodes, to restore antioxidant status in the elderly.
